(19) United States
(12) Patent Application Publication
(10) Pub. No.: US 2026/0315641 A1
(43) Pub. Date: Oct. 15, 2026

(54) FRAGRANCE DIFFUSING DEVICE

(71) Applicant: Guangdong Chiyang Scent Technology Co ., LTD., Foshan (CN)

(72) Inventors: Xiaoyang Gao, Foshan (CN); Xiao Liu, Foshan (CN); Huahui Lao, Foshan (CN); Jie Lan, Foshan (CN); Qunqing Zhu, Foshan (CN)

(21) Appl. No.: 19/303,361

(22) Filed: Aug. 19, 2025

(30) Foreign Application Priority Data
Apr. 10, 2025 (CN) 202510448972.5

(51) Int. Cl. A61L 9/14

(52) U.S. Cl. CPC A61L 9/14; A61L 2209/111; A61L 2209/134

(57) ABSTRACT

A fragrance diffusing device includes: a liquid storage bottle, an air pump, an atomizing head assembly defining an air supply channel and including an atomizing core, a first shell, a second shell, and a third shell. An end of the atomizing head assembly is connected to the liquid storage bottle. The air pump is connected to the air supply channel to supply air to an air outlet opening of the atomizing core. An end of the first shell is connected to the second shell. Another end of the first shell is detachably connected to the third shell. The first shell defines a first accommodation chamber with an open end. The atomizing head assembly connected to the liquid storage bottle enters the first accommodation chamber through the open end. The first, second and third shells together define a mounting space, and the air pump is mounted in the mounting space.

### TECHNICAL FIELD

The disclosure relates to the technical field of fragrance devices, and more particularly to a fragrance diffusing device.

### BACKGROUND

With increasing demands for indoor environmental quality, fragrance diffusing devices, which improve air quality by atomizing aromatic substances, have been widely used in public spaces such as hotels, shopping malls, and offices. Traditional fragrance diffusing devices mainly rely on the siphon principle to transport essential oils, atomize liquid fragrances through atomizing cores, and disperse aromatic substances in the air with the power of air pumps. The typical structure consists of core components such as an atomizing assembly, an essential oil storage bottle, an air pump system, and a device housing.

To achieve better atomization effects and minimize noise, the atomizing assembly is often constructed from multiple parts through step-by-step assembly, and multiple shells are set up to secure the atomizing assembly and the air pump, and to block the noise from the air pump. During the production process, various parts of the atomizing assembly and the shells have to go through multiple separate assembly procedures, resulting in a fragmented structure, complicated assembly, and significantly reduced assembly efficiency of the fragrance diffusing device. Moreover, the complex assembly structure not only increases material costs but also makes it necessary to disassemble the device layer by layer for maintenance, greatly increasing the difficulty of later maintenance.

### SUMMARY

In view of the foregoing, the disclosure aims to provide a fragrance diffusing device to solve the above problem of low assembly efficiency and inconvenient maintenance, which is caused by the need for multiple step-by-step assembly procedures for core components such as the atomizing assembly and the shells.

In order to solve the above problem, the disclosure has following technical solutions.

Specifically, the fragrance diffusing device of the disclosure includes: a liquid storage bottle, an air pump, an atomizing head assembly, a first shell, a second shell, and a third shell. The atomizing head assembly defines an air supply channel, an end of the atomizing head assembly is connected to the liquid storage bottle, and the atomizing head assembly includes an atomizing core. The air pump is connected to the air supply channel to supply air to an air outlet opening of the atomizing core. An end of the first shell is detachably connected to the second shell, or is integrally formed with the second shell. Another end of the first shell is detachably connected to the third shell, and the first shell defines a first accommodation chamber with an open end arranged on an end of the first accommodation chamber. The atomizing head assembly is connected to the liquid storage bottle, enters the first accommodation chamber through the open end, and is fixed in the first accommodation chamber. The first shell, the second shell and the third shell together define a mounting space, and the air pump is mounted in the mounting space.

In an embodiment, the fragrance diffusing device further includes a fan disposed on the second shell and located between the second shell and the third shell.

The first shell defines a first through hole facing towards the fan, the atomizing head assembly defines a spray outlet nozzle and a wind channel, a wind inlet end of the wind channel is connected to the first through hole, a wind outlet end of the wind channel is disposed surrounding the spray outlet nozzle, and the fan is configured to blow wind to the wind inlet end of the wind channel.

The open end is arranged on the end of the first shell connected to the second shell.

In an embodiment, a side of the second shell facing towards the third shell is provided with fixing clips on two sides of the fan, the fixing clips are elastic, and the fan is fixedly disposed between the fixing clips.

In an embodiment, the second shell defines second through holes facing towards the fan, and the second through holes are disposed on the fan, and adjacent to the fixing clips; and/or the side of the second shell facing towards the third shell is provided with limiting members spaced apart around the fan.

In an embodiment, the fragrance diffusing device further includes a fan disposed on the third shell and located between the second shell and the third shell.

The first shell defines a first through hole facing towards the fan, the atomizing head assembly defines a spray outlet nozzle and a wind channel, a wind inlet end of the wind channel is connected to the first through hole, a wind outlet end of the wind channel is disposed surrounding the spray outlet nozzle, and the fan is configured to blow wind to the wind inlet end of the wind channel.

The open end is arranged on the another end of the first shell connected to the third shell.

In an embodiment, the third shell is provided with a first fixing seat connected to the third shell. The first fixing seat is disposed on a side of the first shell and extends towards the second shell. The air pump is suspended on the first fixing seat through flexible support legs.

In an embodiment, an extending direction of the flexible support legs is perpendicular to an inserting direction of the liquid storage bottle. The first shell defines a third through hole, and the air pump is provided with an air outlet tube. The air outlet tube is sleeved with a flexible air conveying tube in a Z shape and is in a sleeve connection with a first end of the flexible air conveying tube. An outer surface of a second end of the flexible air conveying tube is provided with a first snap ring and a second snap ring. The first snap ring and the second snap ring extend along a radial direction of the second end of the flexible air conveying tube. The first snap ring and the second snap ring are spaced apart along an axial direction of the second end of the flexible air conveying tube.

The first snap ring and the second snap ring are tightly fitted on two sides of the third through hole, the second end of the flexible air conveying tube is connected to the first shell, and an air inlet end of the air supply channel is inserted into the second end of the flexible air conveying tube along the axial direction of the second end of the flexible air conveying tube to connect to the air pump.

In an embodiment, the outer surface of the first shell is mounted with multiple liquid level detecting electrodes spaced apart, and the liquid level detecting electrodes are configured to provide detecting signals to the fragrance diffusing device to calculate a liquid level height in the liquid storage bottle according to the detecting signals of the liquid level detecting electrodes.

In an embodiment, the first shell is cylindrical, an outer surface of the first shell is provided with an elongated plane or a recess extending from the third shell to the second shell, and the liquid level detecting electrodes are attached to the elongated plane or the recess.

In an embodiment, the liquid level detecting electrodes are arranged in at least two groups, a number of the liquid level detecting electrodes of each of the at least two groups is in a range of 2-5, the liquid level detecting electrodes of each of the at least two groups are disposed in a straight line along a height direction of the first shell and are spaced apart, the at least two groups have the same number of the liquid level detecting electrodes, the at least two groups are disposed at same heights with the liquid level detecting electrodes in one-to-one correspondence, and the at least two groups are spaced apart along a circumferential direction of the first shell and disposed on an outer surface of the first shell.

In an embodiment, a lowest one of the liquid level detecting electrodes of each of the at least two groups is a reference electrode, the other of the liquid level detecting electrodes of each of the at least two groups are liquid level electrodes.

The fragrance diffusing device is configured to, when detecting the liquid level height, collect a current frequency change Cex of the reference electrode, collect a frequency change Cey′ of a liquid level electrode corresponding to a current liquid level, and then calculate the liquid level height as follows: H=1/K×Cey′/Cex, where K represents a preset coefficient, Cex=Ceb−Cea, Ceb represents a current frequency of the reference electrode, Cea represents an unloaded frequency of the reference electrode, Cey′=Ced−Cec, Ced represents a current frequency of the liquid level electrode corresponding to the current liquid level, and Cec represents an unloaded frequency of the liquid level electrode corresponding to the current liquid level.

The liquid level electrode corresponding to the current liquid level is a candidate one of the liquid level electrodes meeting following conditions: a frequency change Cey′ of the candidate one of the liquid level electrodes is greater than a preset value, and the candidate one of the liquid level electrodes is highest; or the frequency change Cey′ of the candidate one of the liquid level electrodes is greater than the preset value, and a frequency change Cey′ of a next higher one of the liquid level electrodes is less than or equal to the preset value.

In an embodiment, the outer surface of the first shell is fixedly mounted with a liquid level detecting circuit board, and the liquid level detecting electrodes are connected to the liquid level detecting circuit board via wires; the outer surface of the first shell is provided with a second fixing seat, and the second fixing seat is configured to fix a power control board of the fragrance diffusing device; the fragrance diffusing device further includes a main control circuit board, the liquid level detecting circuit board and the power control board are connected to the main control circuit board via wires; and the outer surface of the first shell is integrally formed with wire clips configured to fixing the wires.

In an embodiment, the first shell is cylindrical, the third shell defines an annular groove corresponding to the first shell, and the first shell is inserted into the annular groove and detachably connected to the third shell.

In an embodiment, an outer wall of the another end of the first shell inserted to third shell defines one of a snap groove and a snap latch, and correspondingly, an inner wall of the annular groove is provided with another of the snap groove and the snap latch. The first shell is inserted into the annular groove and rotatable to engage the snap latch and the snap groove.

In an embodiment, the fragrance diffusing device further includes an outer housing. The outer housing includes a tubular main housing body and a cover body. The first shell, the second shell, the third shell, and the liquid storage bottle are inserted into and fixed to the tubular main housing body; and the cover body is connected to the tubular main housing body to enclose the first shell, the second shell and the third shell in the outer housing, the cover body defines an opening corresponding to the open end, and the atomizing head assembly connected to the liquid storage bottle is inserted into the first shell through the opening.

Alternatively, the outer housing includes a middle housing and two side cover plates, the middle housing is elongated and bendable, the middle housing wraps around outer sides of the first shell, the second shell and the third shell to form a rectangular frame, and the two side cover plates are connected to front and rear sides of the rectangular frame respectively; and the middle housing defines an opening corresponding to the open end, and the atomizing head assembly connected to the liquid storage bottle is inserted into the first shell through the opening.

In an embodiment, the outer housing includes three parts, a first part is the tubular main housing body (including the first shell, the second shell and the third shell), a second part is the cover body (which is fitted around the periphery of the tubular main housing body and can be made of metal such as iron sheet, i.e., the middle housing), and a third part is the two side cover plates.

Compared to the related art, the fragrance diffusing device of the embodiments of the disclosure may have following beneficial effects.

The atomizing head assembly connected to the liquid storage bottle may enter the first accommodation chamber through the open end and be fixed inside the first accommodation chamber, and thus the atomizing head assembly, the liquid storage bottle and the first shell form a structural unit, facilitating further installation.

The second shell is connected to the end of the first shell, the another end of the first shell is connected to the third shell, and the first shell, the second shell and the third shell together define the mounting space, and the air pump is mounted in the mounting space. Thus, by simply connecting the first, second, and third shells, the core components of the fragrance diffusing device, including the atomizing head assembly, the liquid storage bottle and the air pump, can be quickly assembled. On one hand, the assembly structure is simple. On the other hand, the atomizing head assembly, the liquid storage bottle and the air pump are organized into a neat structural module, facilitating quick assembly, which greatly improves assembly efficiency; meanwhile, the atomizing head assembly, the liquid storage bottle and the air pump are independent of each other in the structural module, making disassembly and maintenance convenient.

Additionally, the air pump is mounted in the mounting space among the first, second and third shells, making the structural module have a rational spatial layout and a compact appearance design. The respective connection between the two ends of the first shell and the second and third shells ensures the stability of a support structure of the structural module. In addition to accommodating and protecting the atomizing head assembly and the liquid storage bottle, the first shell can also protect the air pump located between the second and third shells from accidental contact during subsequent mounting processes which may cause dislodgement.

### BRIEF DESCRIPTION OF DRAWINGS

Through exemplary embodiments of the disclosure as shown in the accompanying drawings, the above and other objectives, features, and advantages of the disclosure will become clearer. The same reference signs in all the drawings indicate the same parts, and the drawings are not intentionally scaled proportionally according to actual dimensions. The focus is on illustrating the essence of the disclosure.

FIG. 1 illustrates a three-dimensional structural diagram of a fragrance diffusing device according to an embodiment of the disclosure.

FIG. 2 illustrates a first three-dimensional diagram of an inner structure of the fragrance diffusing device according to the embodiment of the disclosure.

FIG. 3 illustrates a second three-dimensional diagram of the inner structure of the fragrance diffusing device according to the embodiment of the disclosure.

FIG. 4 illustrates a third three-dimensional diagram of the inner structure of the fragrance diffusing device according to the embodiment of the disclosure.

FIG. 5 illustrates a fourth three-dimensional diagram of the inner structure of the fragrance diffusing device according to the embodiment of the disclosure.

FIG. 6 illustrates a schematic diagram of an assembly state of a first shell and a second shell according to the embodiment of the disclosure.

FIG. 7 illustrates a schematic diagram of an assembly state of an atomizing head assembly and a liquid storage bottle according to the embodiment of the disclosure.

FIG. 8 illustrates a three-dimensional structural diagram of a third shell according to the embodiment of the disclosure.

FIG. 9 illustrates a schematic diagram of an assembly process of the atomizing head assembly, the liquid storage bottle and the first shell according to the embodiment of the disclosure.

FIG. 10 illustrates a partial structural diagram of a mounting relationship between the liquid storage bottle and the first shell according to the embodiment of the disclosure.

FIG. 11 illustrates an exploded view of an outer housing according to the embodiment of the disclosure.

FIG. 12 illustrates a first schematic diagram of alignment arrangement of liquid level electrodes and reference electrodes according to the embodiment of the disclosure.

FIG. 13 illustrates a second schematic diagram of the alignment arrangement of the liquid level electrodes and the reference electrodes according to the embodiment of the disclosure.

### DESCRIPTION OF REFERENCE SIGNS

100: liquid storage bottle; 200: atomizing head assembly; 201: air supply channel; 202: atomizing core; 203: spray outlet nozzle; 204: wind channel; 300: air pump; 400: first shell; 500: second shell; 600: third shell; 700: fan; 800: outer housing; 801: middle housing; 802: side cover plate; 803: web; 190: plate frame; 191: plate body; 900: mounting space; 301: air outlet tube;
1: snap groove; 2: open end; 3: snap latch; 5: fixing clip; 6: first through hole; 7: second through hole; 8: third through hole; 9: limiting member; 10: first fixing seat; 11: flexible air conveying tube; 12: first snap ring; 13: second snap ring; 14: elongated plane; 15: liquid level detecting electrode; 151: reference electrode; 152: liquid level electrode; 16: second fixing seat; 17: wire clip; 18: annular groove; 19: opening; 20: liquid level detecting circuit board; 21: power control board; 22: main control circuit board; 23: flexible connecting member; 24: seat plate; 25: hanging member; 26: pad; 27: recess; 28: wire; 2021: air outlet opening.

### DETAILED DESCRIPTION OF EMBODIMENTS

A further detailed description of the technical solutions of the disclosure is provided below in conjunction with the accompanying drawings and specific embodiments, in order to enable those skilled in the art to better understand and implement the disclosure. However, the illustrated embodiments are not intended to limit the disclosure. In the embodiments, it should be understood that the terms “longitudinal”, “transverse”, “up”, “down”, “front”, “back”, “left”, “right”, “vertical”, “horizontal”, “top”, “bottom”, “inside”, “outside” and other directional or positional relationships indicated are based on the directional or positional relationships shown in the accompanying drawings, and are only for the convenience of describing the disclosure, rather than indicating or implying that the device or component referred to must have a specific orientation, be constructed and operated in a specific orientation, and therefore cannot be understood as limiting the disclosure.

It should be noted that when a component is considered to be “connected” to another component, it can be directly connected to the another component and integrated with it, or there may be a central component present at the same time. The terms “installation”, “mounting”, “one end”, “another end” and similar expressions used in the disclosure are for illustrative purposes only.

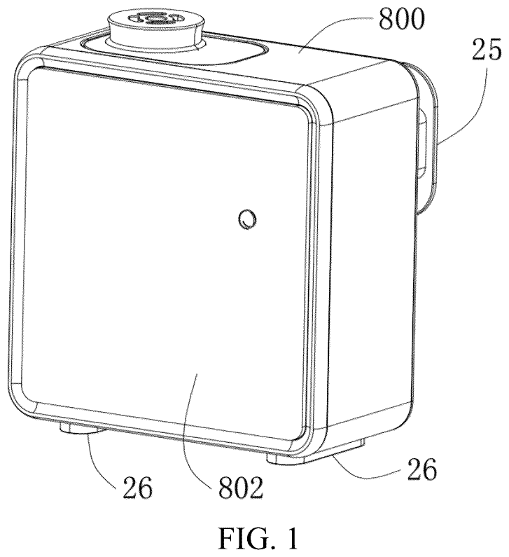

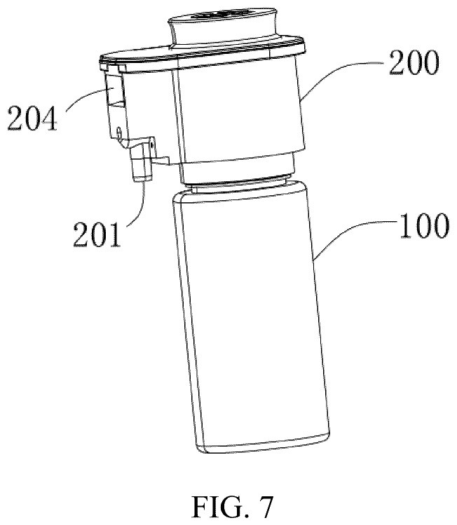

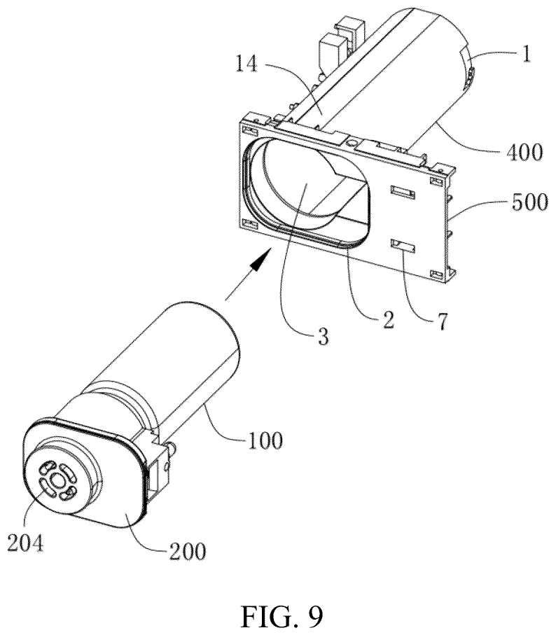

An embodiment of the disclosure provides a fragrance diffusing device, as shown in FIGS. 1-11. The fragrance diffusing device includes: a liquid storage bottle 100, an air pump 300, a first shell 400, a second shell 500, a third shell 600, and an atomizing head assembly 200. The atomizing head assembly 200 defines an air supply channel 201, an end of the atomizing head assembly 200 is connected to the liquid storage bottle 100, and the atomizing head assembly 200 includes an atomizing core 202. The air pump 300 is connected to the air supply channel 201 to supply air to an air outlet opening 2021 of the atomizing core 202. An end of the first shell 400 is integrally formed with the second shell 500, and another end of the first shell 400 is detachably connected to the third shell 600. The first shell 400 defines a first accommodation chamber 3 with an open end 2 arranged on an end of the first accommodation chamber 3. The atomizing head assembly 200 is connected to the liquid storage bottle 100, enters the first accommodation chamber 3 through the open end 2, and is fixed in the first accommodation chamber 3. The first shell 400, the second shell 500 and the third shell 600 together define a mounting space 900, and the air pump 300 is mounted in the mounting space 900. It should be noted that, in an exemplary embodiment, the end of the first shell 400 may be detachably connected to the second shell 500, and the first shell 400 and the second shell 500 are typically made of plastic material.

The atomizing head assembly 200 connected to the liquid storage bottle 100 may enter the first accommodation chamber 3 through the open end 2 and be fixed inside the first accommodation chamber 3, and thus the atomizing head assembly 200, the liquid storage bottle 100 and the first shell 400 form a structural unit, facilitating further installation.

The second shell 500 is connected to the end of the first shell 400, the another end of the first shell 400 is connected to the third shell 600, and the first shell 400, the second shell 500 and the third shell 600 together define the mounting space 900, and the air pump 300 is mounted in the mounting space 900. Thus, by simply connecting the first shell 400, the second shell 500 and the third shell 600, the core components of the fragrance diffusing device, including the atomizing head assembly 200, the liquid storage bottle 100 and the air pump 300, can be quickly assembled. On one hand, the assembly structure is simple. On the other hand, the atomizing head assembly 200, the liquid storage bottle 100 and the air pump 300 are organized into a neat structural module, facilitating quick assembly, which greatly improves assembly efficiency; meanwhile, the atomizing head assembly 200, the liquid storage bottle 100 and the air pump 300 are independent of each other in the structural module, making disassembly and maintenance convenient.

Additionally, the air pump 300 is mounted in the mounting space 900 among the first shell 400, the second shell 500 and the third shell 600, making the structural module have a rational spatial layout and a compact appearance design. The respective connection between the two ends of the first shell 400 and the second shell 500 and the third shell 600 ensures the stability of a support structure of the structural module. In addition to accommodating and protecting the atomizing head assembly 200 and the liquid storage bottle 100, the first shell 400 can also protect the air pump 300 located between the second shell 500 and the third shell 600 from accidental contact during subsequent mounting processes which may cause dislodgement.

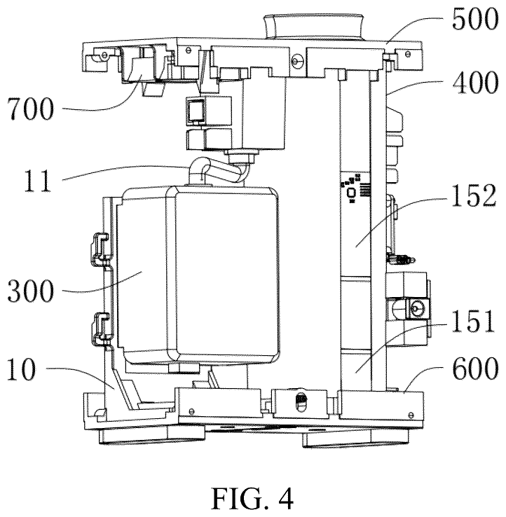

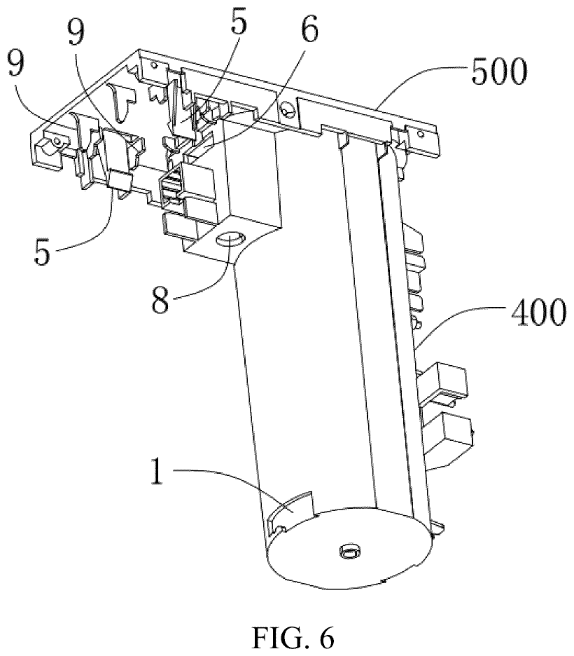

In an exemplary embodiment, as shown in FIG. 4 and FIG. 6, the fragrance diffusing device further includes a fan 700. The fan 700 is disposed on the second shell 500 and located between the second shell 500 and the third shell 600. The first shell 400 defines a first through hole 6 facing towards the fan 700, the atomizing head assembly 200 defines a spray outlet nozzle 203 and a wind channel 204, a wind inlet end of the wind channel 204 is connected to the first through hole 6, a wind outlet end of the wind channel 204 is disposed surrounding the spray outlet nozzle 203, and the fan 700 is configured to blow wind to the wind inlet end of the wind channel 204. The open end 2 is arranged on the end of the first shell 400 connected to the second shell 500.

In the embodiment, the wind (i.e., airflow) blown by the fan 700 may flow to the wind channel 204 through the first through hole 6, and exit from the wind outlet end of the wind channel 204 surrounding the spray outlet nozzle 203, which helps to disperse the atomized particles that emanate from the spray outlet nozzle 203 over a greater distance, thereby expanding the diffusion range of the fragrance diffusing device.

In an exemplary embodiment, as shown in FIG. 6, a side of the second shell 500 facing towards the third shell 600 is provided with fixing clips 5 on two sides of the fan 700, the fixing clips 5 are elastic, and the fan 700 is fixedly disposed between the fixing clips 5. When installing, the fan 700 can be secured by applying slight force to snap it into place between the fixing clips 5 on the two sides of the fan 700, and thereby the fan 700 can be fixed, making the installation convenient and quick.

In an exemplary embodiment, the second shell 500 defines second through holes 7 facing towards the fan 700, and the second through holes 7 are disposed on the fan 700, and adjacent to the fixing clips 5. The second through holes 7 can connect the air around the fan 700, conducive to accelerating the heat dissipation rate of the fan 700.

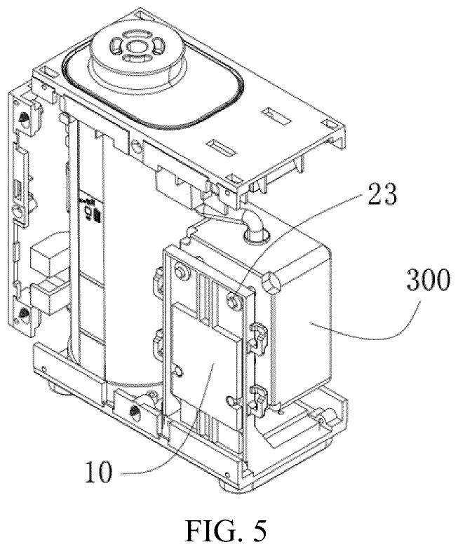

The side of the second shell 500 facing towards the third shell 600 is provided with limiting members 9 spaced apart around the fan 700 to improve the stability of the fan 700 during operating. Specifically, as shown in FIG. 5, ends of the limiting members 9 are chamfered to provide guidance for installation of the fan 700 and enable quick assembly.

In another exemplary embodiment, the fragrance diffusing device further includes: a fan 700 disposed on the third shell 600 and located between the second shell 500 and the third shell 600. The first shell 400 defines a first through hole 6 facing towards the fan 700, the atomizing head assembly 200 defines a spray outlet nozzle 203 and a wind channel 204, a wind inlet end of the wind channel 204 is connected to the first through hole 6, a wind outlet end of the wind channel 204 is disposed surrounding the spray outlet nozzle 203, and the fan 700 is configured to blow wind to the wind inlet end of the wind channel 204. The open end 2 is arranged on the another end of the first shell 400 connected to the third shell 600. That is, the fan 700 can be disposed on the third shell 600 to achieve the above blowing effect.

In an exemplary embodiment, as shown in FIG. 4 and FIG. 5, the third shell 600 is provided with a first fixing seat 10 connected to the third shell 600. The first fixing seat 10 is disposed on a side of the first shell 400 and extends towards the second shell 500. In this embodiment, the first fixing seat 10 is L-shaped. Specifically, the first fixing seat 10 can be connected to the third shell 600 through detachable connecting methods such as screws. The air pump 300 is suspended on the first fixing seat 10 through flexible support legs, which may reduce the vibration and noise generated by collisions with the surroundings during the operation of the air pump 300. During the installation process, due to the large open space between the second shell 500 and the third shell 600, it is possible to first connect the first fixing seat 10 to the third shell 600, then connect the third shell 600 to the first shell 400, and finally secure the air pump 300 to the first fixing seat 10.

In an exemplary embodiment, an extending direction of the flexible support legs is perpendicular to an inserting direction of the liquid storage bottle 100. For example, as shown in FIG. 5, the first fixing seat 10 is provided with flexible connecting members 23 as the flexible support legs. A first end of each flexible connecting member 23 is connected to the air pump 300, and a second end of each flexible connecting member 23 is connected to the first fixing seat 10, making the air pump 300 installed and fixed on the first fixing seat 10 in a suspended state. By utilizing the flexibility of the flexible connecting members 23, the air pump 300 can be securely installed in a “suspended” state on the first fixing seat 10, reducing the vibrations transmitted from the air pump 300 to the first fixing seat 10 and an outer housing 800, thereby minimizing noise and its environmental impact. Consequently, it prevents disturbances to healthy living conditions of people and user inconvenience, thus enhancing the user experience. Specifically, the “suspended” state of the air pump 300 avoids the impact sounds generated by vibrations during operation, which would otherwise result from hard contact with the surrounding component surfaces, effectively lowering the noise level during product operation. In some embodiments, the flexible connecting members 23 are evenly distributed on a side of the air pump 300, ensuring a more uniform force distribution among the flexible connecting members 23, thereby reducing the vibrations transmitted to the first fixing seat 10 from the air pump 300, and further reducing noise.

Moreover, when the air pump 300 is in operation, it generates vibrations. This suspended arrangement through the flexible connecting members 23 can offset a portion of these vibrations, thereby reducing the noise caused by the overall vibration of the product and the vibration between the placement platform during product operation.

In some embodiments of the disclosure, a number of the flexible connecting members 23 is four. The air pump 300 in this embodiment is cuboid-shaped, and each flexible connecting member 23 is evenly distributed at four corners of the air pump 300. Using only four flexible connecting members 23 can achieve vibration damping and noise reduction for the cuboid-shaped air pump 300. This not only saves manufacturing costs but also ensures a more uniform force distribution among the flexible connecting members 23.

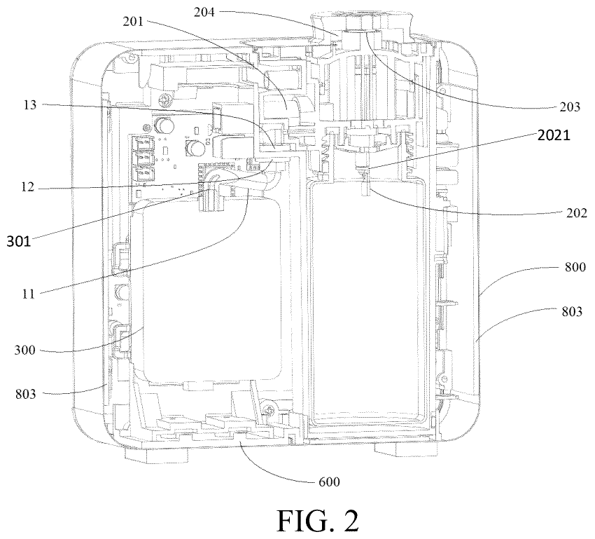

In an exemplary embodiment, as shown in FIGS. 2, 4 and 6, the extending direction of the flexible support legs is perpendicular to the inserting direction of the liquid storage bottle 100. The first shell 400 defines a third through hole 8, and the air pump 300 is provided with an air outlet tube 301. The air outlet tube 301 is sleeved with a flexible air conveying tube 11 in a Z shape and is in a sleeve connection with a first end of the flexible air conveying tube 11. An outer surface of a second end of the flexible air conveying tube 11 is provided with a first snap ring 12 and a second snap ring 13. The first snap ring 12 and the second snap ring 13 extend along a radial direction of the second end of the flexible air conveying tube. The first snap ring 12 and the second snap ring 13 are spaced apart along an axial direction of the second end of the flexible air conveying tube 11.

The first snap ring 12 and the second snap ring 13 are tightly fitted on two sides of the third through hole 8, the second end of the flexible air conveying tube 11 is connected to the first shell 400, and an air inlet end of the air supply channel 201 is inserted into the second end of the flexible air conveying tube 11 along the axial direction of the second end of the flexible air conveying tube 11 to connect to the air pump 300.

In the embodiment, when the air pump 300 is operating and supplying air to the flexible air conveying tube 11, the first end of the Z-shaped flexible air conveying tube 11, due to the reaction force from the second end thereof, will tightly fit over the air outlet tube 301 of the air pump 300 and will not easily come off, ensuring the stability of the air supply.

In an exemplary embodiment, an outer surface of the first shell 400 is mounted with multiple liquid level detecting electrodes 15 spaced apart, and the liquid level detecting electrodes 15 are configured to provide detecting signals to the fragrance diffusing device to calculate a liquid level height in the liquid storage bottle 100 according to the detecting signals of the liquid level detecting electrodes 15.

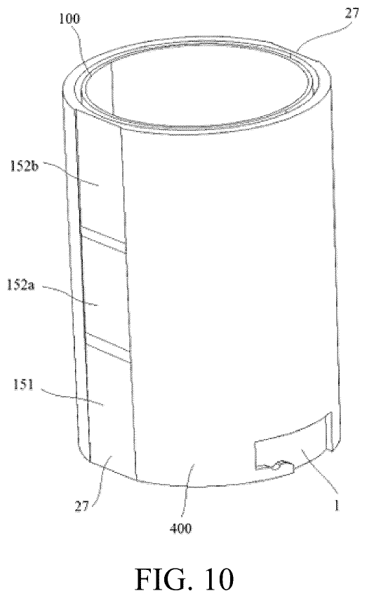

In a further exemplary embodiment, as shown in FIGS. 6 and 10, the first shell 400 is cylindrical. The outer surface of the first shell 400 is provided with an elongated plane 14 or a recess 27 extending from the third shell 600 to the second shell 500, and the liquid level detecting electrodes 15 are attached to the elongated plane 14 or the recess 27. The liquid level detecting electrodes 15 are arranged extending along a liquid level height direction, thereby enabling the measurement of any liquid level height in the liquid storage bottle 100.

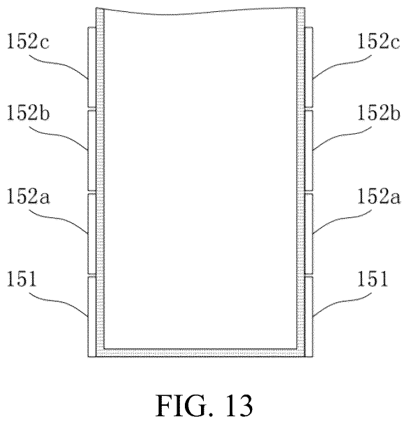

As shown in FIGS. 12-13, the liquid level detecting electrodes 15 are arranged in at least two groups, a number of the liquid level detecting electrodes 15 of each of the at least two groups is in a range of 2-5, the liquid level detecting electrodes 15 of each of the at least two groups are disposed in a straight line along a height direction of the first shell 400 and are spaced apart to avoid mutual interference therebetween. The at least two groups are disposed at same heights with the liquid level detecting electrodes 15 in one-to-one correspondence, and the at least two groups are spaced apart along a circumferential direction of the first shell 400 and disposed on the outer surface of the first shell 400.

In a further exemplary embodiment, a lowest one of the liquid level detecting electrodes 15 of each of the at least two groups is a reference electrode 151, the other of the liquid level detecting electrodes 15 of each of the at least two groups are liquid level electrodes 152. The reference electrode 151 and the liquid level electrodes 152 are non-contact capacitive sensors, which are attached to the outer wall of the liquid storage bottle 100 for use. When the liquid level changes, it causes a frequency change of the electrodes.

The fragrance diffusing device is configured to, when detecting the liquid level height, collect a current frequency change Cex of the reference electrode 151, collect a frequency change Cey′ of a liquid level electrode 152 corresponding to a current liquid level, and then calculate the liquid level height as follows: H=1/K×Cey′/Cex, where K represents a preset coefficient, Cex=Ceb−Cea, Ceb represents a current frequency of the reference electrode 151, Cea represents an unloaded frequency of the reference electrode 151, Cey′=Ced−Cec, Ced represents a current frequency of the liquid level electrode 152 corresponding to the current liquid level, and Cec represents an unloaded frequency of the liquid level electrode 152 corresponding to the current liquid level. It should be noted that, the frequency value of each of the reference electrode 151 and the liquid level electrode 152 can be an average frequency value of the corresponding liquid level detecting electrodes 15 at a corresponding height in the at least two groups.

The liquid level electrode 152 corresponding to the current liquid level is a candidate liquid level electrode meeting following conditions: a frequency change Cey′ of the candidate liquid level electrode is greater than a preset value, and the candidate liquid level electrode is highest; or the frequency change Cey′ of the candidate liquid level electrode is greater than the preset value, and a frequency change Cey′ of a next higher one of the liquid level electrodes 152 (one of the liquid level electrodes next higher than the candidate liquid level electrode) is less than or equal to the preset value.

That is, the candidate liquid level electrode meeting the following conditions is the liquid level electrode 152 corresponding to the current liquid level.
(1) A frequency change Cey′ of the candidate liquid level electrode is greater than the preset value, and the candidate liquid level electrode is the highest one of the liquid level electrodes 152. This candidate liquid level electrode is the liquid level electrode 152 corresponding to the current liquid level.

Referring to FIG. 10, the preset value is 0, the liquid level electrodes 152 include a first liquid level electrode 152a and a second liquid level electrode 152b. The second liquid level electrode 152b is relatively closer to the bottle mouth, therefore, the second liquid level electrode 152b is the highest one of the liquid level electrodes 152. When the frequency change of the second liquid level electrode 152b is greater than 0, it indicates a full bottle state, and thus the second liquid level electrode 152b is the liquid level electrode 152 corresponding to the current liquid level.
(2) The frequency change Cey′ of the candidate liquid level electrode is greater than the preset value, and the frequency change Cey′ of the next higher one of the liquid level electrodes 152 is less than or equal to the preset value. This candidate liquid level electrode is the liquid level electrode 152 corresponding to the current liquid level.

Referring to FIG. 13, when the preset value is 0, a first liquid level electrode 152a, a second liquid level electrode 152b, and a third liquid level electrode 152c are sequentially disposed in that order along a direction from the bottle bottom to the bottle mouth. When the frequency change of the third liquid level electrode 152c≤0, it is indicated that it is not a full bottle state, at this point, when the frequency change of the second liquid level electrode 152b>0, the second liquid level electrode 152b is the liquid level electrode 152 corresponding to the current liquid level.

Also referring to FIG. 10, when the frequency change of the second liquid level electrode 152b≤0, and the frequency change of the first liquid level electrode 152a>0, the first liquid level electrode 152a is the liquid level electrode 152 corresponding to the current liquid level.

Specifically, the frequency change Cey′ of the liquid level electrode 152 corresponding to the current liquid level meet the following condition: Cey′=Ced−Cec, where Ced represents the current frequency of the liquid level electrode 152 corresponding to the current liquid level, and Cec represents the unloaded frequency of the liquid level electrode 152 corresponding to the current liquid level.

For example, when the first liquid level electrode 152a is the liquid level electrode 152 corresponding to the current liquid level, Ced is the detected frequency of the first liquid level electrode 152a.

When the reference electrodes 151 and the liquid level electrodes 152 are arranged in the at least two groups, a number of the liquid level electrodes 152 corresponding to the current liquid level is at least two. Ced is an average value of the current frequencies of the liquid level electrodes 152 corresponding to the current liquid level. For example, when the first liquid level electrodes 152a are the liquid level electrodes 152 corresponding to the current liquid level, Ced is an average value of the current frequencies of the multiple first liquid level electrodes 152a.

Specifically, the current frequency change Cex of the reference electrode 151 meets the following condition: Cex=Ceb−Cea, where Ceb represents the current frequency of the reference electrode 151, and Cea represents the unloaded frequency of the reference electrode 151.

When the reference electrodes 151 and the liquid level electrodes 152 are arranged in the at least two groups, a number of the liquid level electrodes 152 corresponding to the current liquid level is at least two. Ceb is an average value of the current frequencies of the multiple reference electrodes 152.

In the embodiment that the reference electrodes 151 and the liquid level electrodes 152 are arranged in the at least two groups, the at least two groups have the same number of the liquid level electrodes 152, and are disposed at same heights with the liquid level electrodes 152 in one-to-one correspondence. The liquid level electrodes 152 are disposed at same heights. As shown in FIG. 13, The left group has two liquid level electrodes 152 disposed, and the right group also has two liquid level electrodes 152 disposed. The two first liquid level electrodes 152a arranged respectively at left and right are disposed at a same height, and the two reference electrodes 152 arranged respectively at left and right are disposed at a same height. In this embodiment, terms left and right refer to the orientation shown in FIG. 13.

In an exemplary embodiment, an end of the reference electrode 151 is flush with the bottom of the liquid storage bottle 100, and an end of the liquid level electrode 152 is flush with the top of the liquid storage bottle 100, thus ensuring accurate measurement of the liquid level from the bottom to the top of the liquid storage bottle 100. The liquid level electrode 152 and the reference electrode 151 are disposed along a height direction of the liquid storage bottle 100, making a segmented electrode arrangement on the liquid storage bottle 100. This design prevents the electrodes from being too long and reduces the risk of increased error due to the large surface area of the electrodes. By using a distributed electrode configuration, the surface area of each electrode is minimized, thereby reducing error.

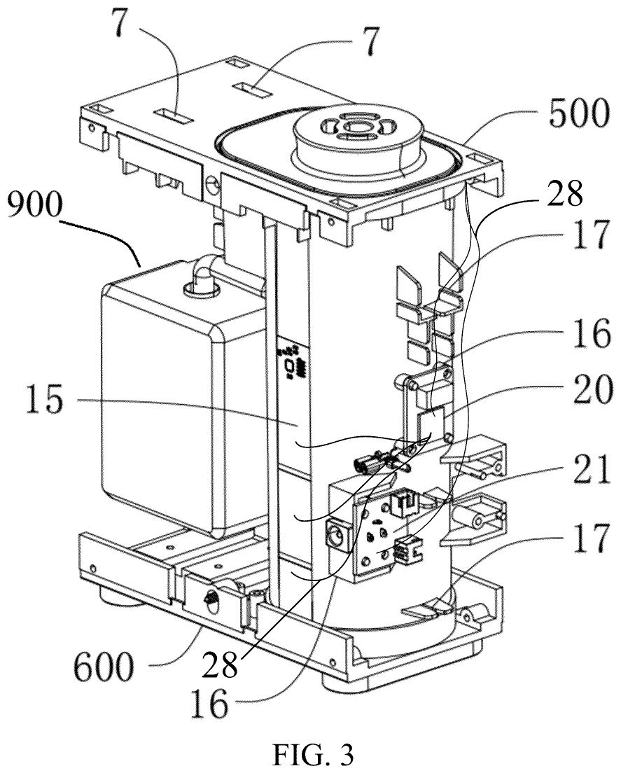

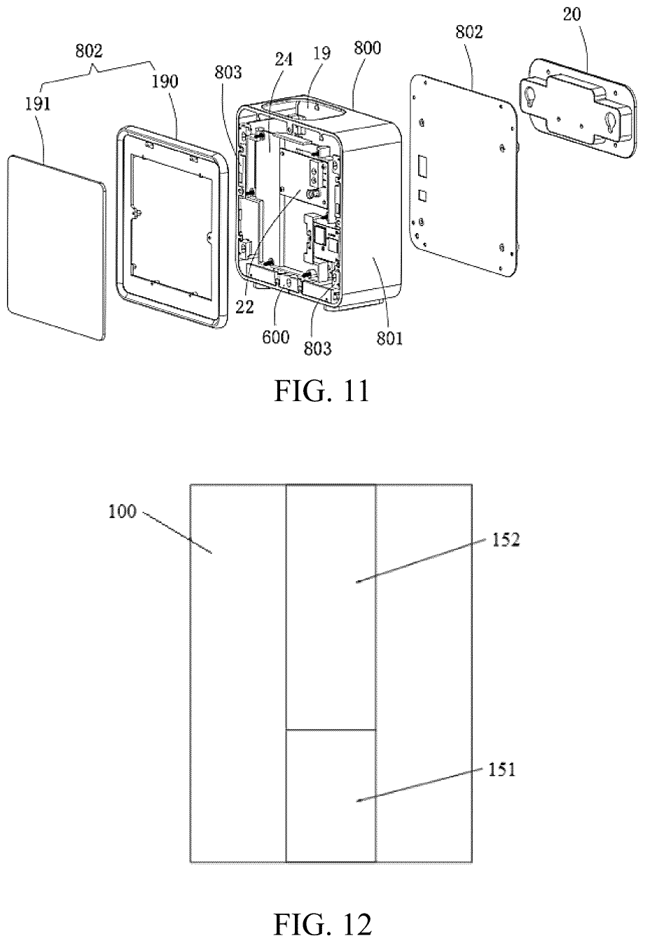

In an exemplary embodiment, as shown in FIGS. 3 and 11, the outer surface of the first shell 400 is fixedly mounted with a liquid level detecting circuit board 20, and the liquid level detecting electrodes 15 are connected to the liquid level detecting circuit board 20 via wires 28. The outer surface of the first shell 400 is provided with a second fixing seat 16, and the second fixing seat 16 is configured to fix a power control board 21 of the fragrance diffusing device; the fragrance diffusing device further includes a main control circuit board 22, the liquid level detecting circuit board 20 and the power control board 21 are connected to the main control circuit board 22 via wires 28; and the outer surface of the first shell 400 is integrally formed with multiple wire clips 17 configured to fixing the wires 28, ensuring that the wires 28 connected to the control boards are neatly arranged and orderly, making the wiring clear and orderly, and avoiding clutter. The specific structure of the second fixing seat 16 is not specifically defined and can take various forms, allowing for adaptive changes according to the objects to be fixed.

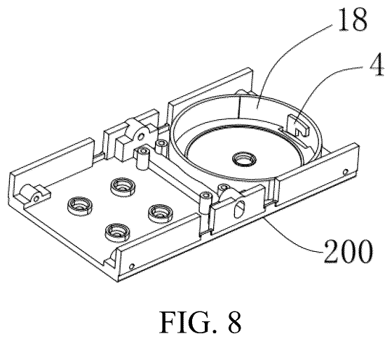

In an exemplary embodiment, as shown in FIGS. 6 and 8, the first shell 400 is cylindrical. The third shell 600 defines an annular groove 18 corresponding to the first shell 400, and the first shell 400 is inserted into the annular groove 18 and detachably connected to the third shell 600. In a further exemplary embodiment, an outer wall of the another end of the first shell 400 inserted to third shell 600 defines one of a snap groove 1 and a snap latch 4, and correspondingly, an inner wall of the annular groove 18 is provided with another of the snap groove 1 and the snap latch 4. The first shell 400 is inserted into the annular groove 18 and then rotated to engage the snap latch 4 and the snap groove 1. This type of connection allows for easy and convenient installation.

Based on the above embodiments, in an exemplary embodiment, the fragrance diffusing device further includes an outer housing 800. The outer housing 800 can be cylindrical or tubular, or rectangular as shown in FIGS. 1-2.

The outer housing 800 includes a tubular main housing body and a cover body. The first shell 400, the second shell 500, the third shell 600, and the liquid storage bottle 100 are inserted into and fixed to the tubular main housing body. The cover body is connected to the tubular main housing body to enclose the first shell 400, the second shell 500 and the third shell 600 in the outer housing 800, the cover body defines an opening 19 corresponding to the open end 2, and the atomizing head assembly 200 connected to the liquid storage bottle 100 is inserted into the first shell 400 through the opening 19.

Alternatively, as shown in FIG. 11, the outer housing 800 includes a middle housing 801 and two side cover plates 802, and the middle housing 801 is elongated and bendable. The outer housing 800 can be made of a highly ductile sheet metal, which is lightweight and less prone to damage. The middle housing 801 wraps around outer sides of the first shell 400, the second shell 500 and the third shell 600 to form a rectangular frame. The two side cover plates 802 are connected to front and rear sides of the rectangular frame respectively. The middle housing 801 defines an opening 19 corresponding to the open end 2, and the atomizing head assembly 200 connected to the liquid storage bottle 100 is inserted into the first shell 400 through the opening 19. It should be noted that, a longitudinal cross-section of the rectangular frame formed by the middle housing 801 is generally rectangular in shape, but it is not a strict right-angled rectangle. The corners can be designed with rounded or chamfered transitions.

In the embodiment, after the atomizing head assembly 200, the liquid storage bottle 100, and the air pump 300 are assembled to a module, the outer housing 800 that can be fitted around the outer periphery of the assembled module is provided. On one hand, this simplifies the installation of the outer housing 800, further enhancing the assembly efficiency. On the other hand, it also facilitates the customized settings of the front and rear side cover plates 802 of the assembled module, meeting the diverse needs of customers regarding the side cover plates 802 or the appearance of the fragrance diffusing device, thereby increasing the versatility of the fragrance diffusing device.

In a further exemplary embodiment, as shown in FIG. 11, a web 803 is provided inside the middle housing 801, extends along an annular direction of the middle housing 801, and is disposed between the second shell 500 and the third shell 600. The side cover plates 802 are connected to the web 803. The web 803 can enhance the impact resistance and deformation resistance of the middle housing 801 and the outer housing 800. Connecting the side cover plates 802 to the web 803 can also increase the stability of the side cover plates 802. Of course, optionally, the side cover plates 802 may also be connected separately to the second shell 500, the third shell 600, and the web 803 located therearound.

Furthermore, each side cover plate 802 includes a plate frame 190 and a plate body 191. The plate frame 190 is connected to the second shell 500, the third shell 600, and the web 803. The plate frame 190 is adhesively connected to the plate body 191. This arrangement avoids the dilemma of connection strength and plate integrity that would arise if the plate body 191 were directly connected to the second shell 500, the third shell 600, and the web 803, thus greatly facilitating the selection of materials and types for the plate body 191. For example, mirror-like panels and liquid crystal display panels can be easily chosen. Moreover, in this embodiment, for touch screen panels that require a touch control circuit board, a seat plate 24 for fixing the touch control circuit board can be provided using the plate frame 190. This facilitates the rational layout of various control boards, including the touch control circuit board, and also improves the utilization of the internal space of the outer housing 800.

It should be noted that the fragrance diffusing device in this embodiment can be used by placing it directly on a flat surface or by hanging it on a wall. Specifically, a hanging member 25 can be provided on one of the side cover plates 802 to fix the fragrance diffusing device on the wall; alternatively, pads 26 which are sound-damping and vibration-absorbing can be provided at the bottom of the outer housing 800.

In the specification, unless otherwise specified and limited, the term “on” or “under” with respect to a first feature relative to a second feature may mean that the first and second features are in direct contact, or that the first and second features are in indirect contact through an intermediate medium. Moreover, the terms “above,” “over,” and “on top of” may indicate that the first feature is directly above or diagonally above the second feature, or simply that the first feature has a higher horizontal level than the second feature. The terms “below,” “under,” and “beneath” may indicate that the first feature is directly below or diagonally below the second feature, or simply that the first feature has a lower horizontal level than the second feature.

In the description of this specification, the reference terms such as “exemplary embodiments”, “further embodiments”, “other embodiments”, or “specific examples” means that the specific features, structures, materials, or characteristics described in conjunction with the embodiments or examples are included in at least one embodiment or example of the disclosure. In this specification, the schematic expressions of the above terms do not necessarily refer to the same embodiments or examples. Moreover, the specific features, structures, materials, or characteristics described can be combined in any one or more embodiments or examples in an appropriate manner. In addition, those skilled in the art can combine the different embodiments or examples described in this specification, as well as the features of different embodiments or examples, without conflicting with each other.

Although the embodiments of the disclosure have been shown and described above, it can be understood that the above embodiments are exemplary and should not be construed as limiting the disclosure. Those skilled in the art may make changes, amendments, substitutions, and variations to the above embodiments within the scope of the disclosure.

What is claimed is:

1. A fragrance diffusing device, comprising: a liquid storage bottle, an air pump, an atomizing head assembly, a first shell, a second shell, and a third shell;
wherein the atomizing head assembly defines an air supply channel, an end of the atomizing head assembly is connected to the liquid storage bottle, and the atomizing head assembly comprises an atomizing core; and the air pump is connected to the air supply channel to supply air to an air outlet opening of the atomizing core; and
wherein an end of the first shell is detachably connected to the second shell, or is integrally formed with the second shell; another end of the first shell is detachably connected to the third shell, and the first shell defines a first accommodation chamber with an open end arranged on an end of the first accommodation chamber; the atomizing head assembly is connected to the liquid storage bottle, enters the first accommodation chamber through the open end, and is fixed in the first accommodation chamber; and the first shell, the second shell and the third shell together define a mounting space, and the air pump is mounted in the mounting space.

2. The fragrance diffusing device as claimed in claim 1, further comprising: a fan disposed on the second shell and located between the second shell and the third shell;
wherein the first shell defines a first through hole facing towards the fan, the atomizing head assembly defines a spray outlet nozzle and a wind channel, a wind inlet end of the wind channel is connected to the first through hole, a wind outlet end of the wind channel is disposed surrounding the spray outlet nozzle, and the fan is configured to blow wind to the wind inlet end of the wind channel; and
wherein the open end is arranged on the end of the first shell connected to the second shell.

3. The fragrance diffusing device as claimed in claim 2, wherein a side of the second shell facing towards the third shell is provided with fixing clips on two sides of the fan, the fixing clips are elastic, and the fan is fixedly disposed between the fixing clips.

4. The fragrance diffusing device as claimed in claim 3, wherein the second shell defines second through holes facing towards the fan, and the second through holes are disposed on the fan, and adjacent to the fixing clips; and/or
wherein the side of the second shell facing towards the third shell is provided with limiting members spaced apart around the fan.

5. The fragrance diffusing device as claimed in claim 1, wherein the first shell is cylindrical, the third shell defines an annular groove corresponding to the first shell, and the first shell is inserted into the annular groove and detachably connected to the third shell.

6. The fragrance diffusing device as claimed in claim 5, wherein an outer wall of the another end of the first shell inserted to third shell defines one of a snap groove and a snap latch, and correspondingly, an inner wall of the annular groove is provided with another of the snap groove and the snap latch; and
wherein the first shell is inserted into the annular groove and rotatable to engage the snap latch and the snap groove.

7. The fragrance diffusing device as claimed in claim 1, further comprising: an outer housing,
wherein the outer housing comprises a tubular main housing body and a cover body; the first shell, the second shell, the third shell, and the liquid storage bottle are inserted into and fixed to the tubular main housing body; and the cover body is connected to the tubular main housing body to enclose the first shell, the second shell and the third shell in the outer housing, the cover body defines an opening corresponding to the open end, and the atomizing head assembly connected to the liquid storage bottle is inserted into the first shell through the opening; or
wherein the outer housing comprises a middle housing and two side cover plates, the middle housing is elongated and bendable, the middle housing wraps around outer sides of the first shell, the second shell and the third shell to form a rectangular frame, and the two side cover plates are connected to front and rear sides of the rectangular frame respectively; and the middle housing defines an opening corresponding to the open end, and the atomizing head assembly connected to the liquid storage bottle is inserted into the first shell through the opening.

8. The fragrance diffusing device as claimed in claim 1, wherein an outer surface of the first shell is mounted with liquid level detecting electrodes spaced apart, and the liquid level detecting electrodes are configured to provide detecting signals to support the fragrance diffusing device to calculate a liquid level height in the liquid storage bottle according to the detecting signals of the liquid level detecting electrodes.

9. A fragrance diffusing device, comprising: a liquid storage bottle, an air pump, an atomizing head assembly, and a first fixing seat;
wherein the atomizing head assembly defines an air supply channel, an end of the atomizing head assembly is connected to the liquid storage bottle, and the atomizing head assembly comprises an atomizing core; the air pump is connected to the air supply channel to supply air to an air outlet opening of the atomizing core; and the air pump is suspended on the first fixing seat through flexible support legs.

10. The fragrance diffusing device as claimed in claim 9, further comprising: a first shell, a second shell and a third shell, wherein an end of the first shell is connected to the second shell, and the first shell defines a first accommodation chamber with an open end defined on an end of the first accommodation chamber; the atomizing head assembly is connected to the liquid storage bottle, enters through the open end and is fixed in the first accommodation chamber; the first shell, the second shell and the third shell together define a mounting space, and the air pump is mounted in the mounting space; and the first fixing seat is connected to the third shell, is disposed on a side of the first shell and extends towards the second shell.

11. The fragrance diffusing device as claimed in claim 10, wherein an extending direction of the flexible support legs is perpendicular to an inserting direction of the liquid storage bottle; the first shell defines a third through hole, and the air pump is provided with an air outlet tube; the air outlet tube is sleeved with a flexible air conveying tube in a Z shape and is in a sleeve connection with a first end of the flexible air conveying tube; an outer surface of a second end of the flexible air conveying tube is provided with a first snap ring and a second snap ring; the first snap ring and the second snap ring extend along a radial direction of the second end of the flexible air conveying tube; and the first snap ring and the second snap ring are spaced apart along an axial direction of the second end of the flexible air conveying tube; and
wherein the first snap ring and the second snap ring are tightly fitted on two sides of the third through hole, the second end of the flexible air conveying tube is connected to the first shell, and an air inlet end of the air supply channel is inserted into the second end of the flexible air conveying tube along the axial direction of the second end of the flexible air conveying tube to connect to the air pump.

12. A fragrance diffusing device, comprising: a liquid storage bottle, an air pump, an atomizing head assembly, a first shell, and a second shell;
wherein the atomizing head assembly defines an air supply channel, an end of the atomizing head assembly is connected to the liquid storage bottle, and the atomizing head assembly comprises an atomizing core; and the air pump is connected to the air supply channel to supply air to an air outlet opening of the atomizing core; and
wherein an end of the first shell is connected to the second shell, the first shell defines a first accommodation chamber, the liquid storage bottle is accommodated in the first accommodation chamber, the first shell is mounted with liquid level detecting electrodes spaced apart, and the liquid level detecting electrodes are configured to provide detecting signals to support the fragrance diffusing device to calculate a liquid level height in the liquid storage bottle according to the detecting signals of the liquid level detecting electrodes.

13. The fragrance diffusing device as claimed in claim 12, wherein an open end is arranged on an end of the first accommodation chamber; and the atomizing head assembly is connected to the liquid storage bottle, enters through the open end and is fixed in the first accommodation chamber.

14. The fragrance diffusing device as claimed in claim 12, further comprising: a third shell, wherein the first shell, the second shell and the third shell together define a mounting space, and the air pump is mounted in the mounting space.

15. The fragrance diffusing device as claimed in claim 14, wherein the first shell is cylindrical, an outer surface of the first shell is provided with an elongated plane or a recess extending from the third shell to the second shell, and the liquid level detecting electrodes are attached to the elongated plane or the recess.

16. The fragrance diffusing device as claimed in claim 12, wherein the liquid level detecting electrodes are arranged in at least two groups, a number of the liquid level detecting electrodes of each of the at least two groups is in a range of 2-5, the liquid level detecting electrodes of each of the at least two groups are disposed in a straight line along a height direction of the first shell and are spaced apart, the at least two groups have a same number of the liquid level detecting electrodes, the at least two groups are disposed at same heights with the liquid level detecting electrodes in one-to-one correspondence, and the at least two groups are spaced apart along a circumferential direction of the first shell and disposed on an outer surface of the first shell.

17. The fragrance diffusing device as claimed in claim 16, wherein a lowest one of the liquid level detecting electrodes of each of the at least two groups is a reference electrode, the other of the liquid level detecting electrodes of each of the at least two groups are liquid level electrodes;
wherein the fragrance diffusing device is configured to, when detecting the liquid level height, collect a current frequency change Cex of the reference electrode, collect a frequency change Cey′ of a liquid level electrode corresponding to a current liquid level, and then calculate the liquid level height as follows: H=1/K×Cey′/Cex, where K represents a preset coefficient, Cex=Ceb−Cea, Ceb represents a current frequency of the reference electrode, Cea represents an unloaded frequency of the reference electrode, Cey′=Ced−Cec, Ced represents a current frequency of the liquid level electrode corresponding to the current liquid level, and Cec represents an unloaded frequency of the liquid level electrode corresponding to the current liquid level; and
the liquid level electrode corresponding to the current liquid level is a candidate one of the liquid level electrodes meeting following conditions: a frequency change Cey′ of the candidate one of the liquid level electrodes is greater than a preset value, and the candidate one of the liquid level electrodes is highest; or the frequency change Cey′ of the candidate one of the liquid level electrodes is greater than the preset value, and a frequency change Cey′ of a next higher one of the liquid level electrodes is less than or equal to the preset value.

18. The fragrance diffusing device as claimed in claim 12, wherein an outer surface of the first shell is fixedly mounted with a liquid level detecting circuit board, and the liquid level detecting electrodes are connected to the liquid level detecting circuit board via wires; the outer surface of the first shell is provided with a second fixing seat, and the second fixing seat is configured to fix a power control board of the fragrance diffusing device; the fragrance diffusing device further comprises a main control circuit board, the liquid level detecting circuit board and the power control board are connected to the main control circuit board via wires; and the outer surface of the first shell is integrally formed with wire clips configured to fixing the wires.

19. The fragrance diffusing device as claimed in claim 12, further comprising: a third shell, wherein the end of the first shell is detachably connected to the second shell, or is integrally formed with the second shell; and another end of the first shell is detachably connected to the third shell.
